(19) United States
(12) Patent Application Publication
(10) Pub. No.: US 2004/0082008 A1
(43) Pub. Date: Apr. 29, 2004

(54) Method for diagnosing and monitoring of vegetative nervous system state and for using in treating of diseases and states featured by hormone disorder and device for realizing the method

(76) Inventors: Valery A. Naumov, St Petersburg (RU); Iouri Z. Iline, St. Petersburg (RU); Tauno O. Virtanen, Helsinki (FI)

Correspondence Address: Ernest I Gifford, Suite 400, 280 North Old Woodward Avenue, Birmingham, MI, 48009, US

(21) Appl. No.: 10/250,471

(22) PCT Filed: Jan. 4, 2002
(86) PCT No.: PCT/FI02/00007

(30) Foreign Application Priority Data
Jan. 9, 2001 (RU) 2001100627

(51) Int. Cl. G01N 33/53; G01N 33/567

(52) U.S. Cl. 435/7.2

(57) ABSTRACT

A method and an apparatus are disclosed for diagnosing, monitoring and for using in treating drugs and toxic substances abuse, alcoholism, drugs and other substances intoxication and hormone disorder due to a disease or natural processes in human organism. Vegetative nervous system (VNS) strain factors (VNSSF) determined according to the present invention have been found to be, along with other vascular (cardiovascular) parameters, a reliable indicator of VNS state (hormone order or disorder). VNSSF determined according to the present invention and/or said parameters can be compared against, respectively, a predetermined VNSSF and/or the said parameters values bands or against their cutoff values or against previously determined VNSSF and/or the said parameters values for the same patient. A comparison against the said predetermined values bands and/or with the said cutoff values helps to foster a diagnosis of normal VNS state or of strained VNS state, the latter may be caused by drug and toxic substances abuse, alcoholism, drugs and other substances intoxication and hormone disorder due to disease or natural processes in human organism. A comparison against previously determined VNSSF and/or the said parameters values for the same patient can be used for monitoring a patient's state and as a tool in treating of diseases and states featured by hormone disorder.

### TECHNICAL FIELD OF THE INVENTION

In accordance with the present invention, a method and a device are provided for diagnosing, monitoring and for using in treating diseases and states featured by hormone disorder, and more specifically a method is provided for diagnosing of patients as having Vegetative nervous system (VNS) state corresponding to drug and toxic substances abuse, alcoholism, drugs and other substances intoxication and hormone disorder due to disease or natural processes in human organism and as having VNS state corresponding to absence of these diseases and states.

### BACKGROUND OF THE INVENTION

Some diseases have a feature of strained Vegetative nervous system (VNS) state (by hormone disorder). Diagnosing of VNS strained state can serve as a first step in diagnosing of diseases and states featured by hormone disorder. This first step at least separates patients into two groups: a group consisting of those with unstrained VNS and a group consisting of those with strained VNS, the latter being an indicator of corresponding diseases and states probable presence and of a necessity to do further more specific tests to diagnose these diseases and states. Quantitative estimation of VNS strain can serve for monitoring of patient's VNS state, this monitoring can be used in Its turn in treating of patients with diseases and states featured by hormone disorder to estimate efficiency of treating methods and medicines used if this efficiency is indicated by increasing or decreasing of VNS strain.

A special interest is that for a method and device requiring several minutes to detect drug and toxic substances abuse, alcoholism, drugs and other substances intoxication at workplace or at pre-employment search in case the said diseases and states are intolerable. Those skilled in the art know various drug and their traces tests from hair, saliva and urine analyses and corresponding kits to other complicated, time consuming and expensive methods. Devices for these analyses are also known. As a rule, simplest and thus cheap methods and devices are selective and not very reliable, thus they require series tests for various substances and sometimes other more reliable confirming methods and devices using. Thus, complete tests cost is increased. The said is especially noticeable in case of mass tests. Cost of sophisticated methods and devices is a question according to definition.

The said above can be easily extended for other diseases and states.

For those skilled in the art, various methods and devices for VNS state estimation are known. Accurate and reliable results are obtained by biochemical analysis of catecholamides in a body liquid phase (A. G. Resnikov, “The methods of hormones determination”, Naukova Dumka, Kiew, 1980). Being a powerful diagnosing means, this method is complex, much labor and time consuming and expensive. The same can be said about a device used in this method.

Nowadays, non-invasive methods and devices are preferable, sometimes being the only ones tolerating the conditions and requirements of screening.

The present invention was stimulated by a call for a non-invasive method and device featured by low cost, several minutes required for test and at least giving a possibility to take out of further more sophisticated methods tests those who have VNS state with no correspondence to diseases and states featured by strained VNS (hormone disorder).

Therefore one of the present invention objects is to provide a method and a device for VNS state estimation featured by quantitative results data, the latter being in absolute values and with probability ascribed.

### SUMMARY OF THE INVENTION

In accordance with the present invention, the method is provided for diagnosing, monitoring and for using in treating of drugs and toxic substances abuse, alcoholism, drugs and other substances intoxication and hormone disorder due to a disease or natural processes in human organism comprising the steps of (1) gathering Vegetative Nervous System (VNS) state information through a non-invasive means from a reference group (RG1) consisting of persons with predetermined absence of drugs and toxic substances abuse, alcoholism, drugs and other substances intoxication and hormone disorder due to a disease or natural processes in human organism and reference groups (RG2) of patients with predetermined presence of drugs and toxic substances abuse, drugs and other substances intoxication and hormone disorder due to a disease or natural processes in human organism, (2) determining the VNS strain factors (VNSSF) from the gathered VNS state information and/or determining other vascular (cardiovascular) parameters, (3) determining the VNSSF and/or the said parameters values bands corresponding to the groups of step 1 above with inherent cutoff values limiting the bands, (4) comparing of the patient's VNSSF and/or the said parameters values against that within the bands of step (3) above, against limiting (cutoff) VNSSF and/or the said parameters values of these bands and against the patient's VNSSF and/or the said parameters values determined earlier, and (5) diagnosing the patient as belonging at least potentially to at least one of the groups (RG1 or RG2) of step (1) above or as having increasing or decreasing VNS strain in time.

In accordance with the present invention, a non-invasive device for diagnosing, monitoring and for using in treating of diseases and states, in particular of drugs and toxic substances abuse, alcoholism, drugs and other substances intoxication and hormone disorder due to a disease or natural processes in human organism, said device comprising (1) a non-invasive means measuring vascular (cardiovascular) parameters characterizing VNS state, (2) analysis means for calculating of VNSSF values from the measured vascular (cardiovascular) parameters values, (3) analysis means for calculating (determination) of VNSSF and/or the said parameters values bands corresponding to different measurements series with inherent cutoff values limiting the bands, (4) analysis means for comparing of the patient's VNSSF and/or the said parameters values against that within the bands in (3) above, against limiting (cutoff) VNSSF and/or the said parameters values of these bands and against the patient's VNSSF and/or the said parameters values determined earlier, and (5) analysis means for diagnosing the patient as corresponding at least potentially to at least one of the said measurements series or as having increasing or decreasing VNS strain in time.

The method has a feature of increasing its selectivity and probability of true results obtaining along with collection of statistical data for correlation of parameters measured and/or determined according to the present invention and of diagnosing results obtained by other more sophisticated methods. In other words, the method according to the present invention has a feature of self-perfection just due to its using.

One of the objects of the present invention is a method for diagnosing of drugs and toxic substances addiction, alcoholism, drugs and other substances intoxication and hormone disorder due to a disease or natural processes in human organism or to at least for separation of persons Into two groups—those with strained VNS state and unstrained VNS state corresponding to drugs and toxic substances addiction, alcoholism, drugs and other substances intoxication and hormone disorder due to a disease or natural processes in human organism and to absence of the said diseases and states, respectively.

The method according to the present invention can be used to diagnose with some universal cutoff value of VNSSF and/or of other parameters characterizing VNS state, but such an approach can be accompanied by increasing of diagnosing mistake probability or by false identification a patient as belonging to a group to which he does not belong (depending upon a particular cutoff value chosen). The said is due to the following fact: Statistical data for RG1 show that VNSSF average and maximal values can differ for populations of different countries and even of different climatic zones regions of large territories countries due to genetic features and different prevalent food, living standards and health care. Thus, to increase the accuracy of the method, statistical data for RG1 is desirable to be gathered for the country (region) where the method is planned to be used. The said is not valid, however, for drug abuse and drug users groups, because it was found that drugs impact upon VNS (causing hormones disorder) is rather heavy to push VNS to evidently strained state never mind hypothetical unstrained state for drug abuse and drug users.

Further object of the present invention is a method and a device with quantitative estimation of VNS state, giving a possibility to determine a probability for a patient with a definite VNS state quantitative estimate to correspond to one of the above groups.

The probability thus determined can be used to meet a method and/or a device according to the present invention to users requirements to the results of diagnosing or separation of patients into two groups said above. Indeed, shifting of a determined cutoff value in direction to the band for RG1 will lead to decreasing of strained VNS state to be determined as unstrained probability and to increasing of false diagnosing of strained VNS state probability, and visa versa. Consider as an example a practical use of the said separation into drug abuse and drug-free persons screening. Normally, the aim is to determine during several seconds or minutes persons with strained VNS state; false unstrained VNS diagnosing probability is to be near zero. These persons are said to be drug-free and are not tested further by other screening methods or devices or by other confirming methods or devices being exact diagnosing means but possessing no advantages set inherent to a method and a drvice according to the present invention (fast&cheap and near-zero false negative results probability being a feature of a special interest for tests at workplaces). Using of a method and a device according to the invention saves time and money. The more is the said cutoff value shift, the more the requirement of low false negative result probability is satisfied. From the other hand, the more is the said cutoff value shift, the less share of tested persons is said to be drug-free, thus the less is overall money and time saving (but the less is a probability to miss a drug abuse). And visa versa. A compromise choice is after the method and a device according to the present invention users specific demand.

Being used prior to test methods/devices possessing unsatisfactory false negative or false positive test results probability, a method/device according to the present invention made to have near-zero corresponding false results probability improves overall corresponding false results probability. Indeed, after separation into two above said groups, only a part of all tested persons is then tested with unsatisfactory false results probability.

Further object of the present invention is a method/device to be used in treatment of diseases and states featured by strained VNS for treatment methods and medicines used efficiency estimation in case a progress or regress can be estimated by a response of VNS state to treatment methods and medicines.

Further object of the present invention is a method/device, which uses, along with other vascular (cardiovascular) parameters characterizing VNS state, an invented VNSSF, found to be a reliable quantitative parameter of VNS strain (hormone disorder). VNSSF can be determined using data received from different principles sensors measuring vascular (cardiovascular) parameters, including blood volume (BV) and pressure (BP), blood volume and pressure variation velocity (BV/dt & BP/dt) and blood volume and pressure variation acceleration (BV/dt2 & BP/dt2). A particular parameter choice made to achieve maximal sensitivity depends upon the sensors in non-invasive means measuring vascular (cardiovascular) parameters) to be used with a particular tests conditions and other factors in mind. It is evident also for those skilled in the art that particular practical sensors characteristics (frequency passband, etc.) can result in different parameters values determined directly from, for example, BP and BP/dt sensors and from one of the said sensors with following differentiation or integration. Thus, to improve accuracy of the method, VNSSF is better to be determined for a particular sensor type and a particular signal from the sensor processing.

Further object of the present invention is a device requiring no separate means for analog-to digital transformation and for dc power supply to amplifying means. These functions are after computer.

Further object of the present invention is a method/device excluding human element mistakes due a possibility of computerized data treatment and diagnosing.

These and other features will become apparent to those skilled in the art upon a review of the detailed description of a preferred embodiment of the present invention presented below, in conjunction with the drawings presented herewith.

### BRIEF DESCRIPTION OF THE DRAWINGS

FIG. 1 is BP/dt vs. time response typical for persons with unstrained VNS state.

FIG. 2 is BP/dt vs. time response typical for persons with strained VNS state.

FIG. 3 is VNSSF value distribution for drug-free and drugs abuse groups.

FIG. 4 is VNSSF value probabilities to correspond to drug-free and to drugs abuse.

FIG. 5 is VNSSF integral probabilities for drug-free and for drugs abuse.

FIG. 6 is a device according to the present invention.

FIG. 7 is a device according to preferred embodiment of the present invention.

### DETAILED DESCRIPTION OF THE PREFERRED EMBODIMENT

Consider working example and experimental results. This example concerns drug screening tests but the method/device according to the present invention using can be readily expanded on diagnosing of other diseases and states featured by strained VNS state (hormone disorder).

Many years practice proved that in case of hormone disorder (strained VNS state), a correlation of left ventricle and aorta contribution to blood flow varies in favor of the latter when compared with that for RG1. It could be noticed, in particular, by analyzing BV vs time response measured by photoplethysmographic sensor affixed to a finger tip or other part of human body. However, this effect of difference between vascular (cardiovascular) parameters responses for strained and unstrained VNS was noticed to be more pronounced in case of using differential pressure measuring sensors with output signal corresponding to BP/dt.

Preferred embodiment of the present invention is described for a case of practical drugs abuse screening (or prescreening if some other screening tests are made further).

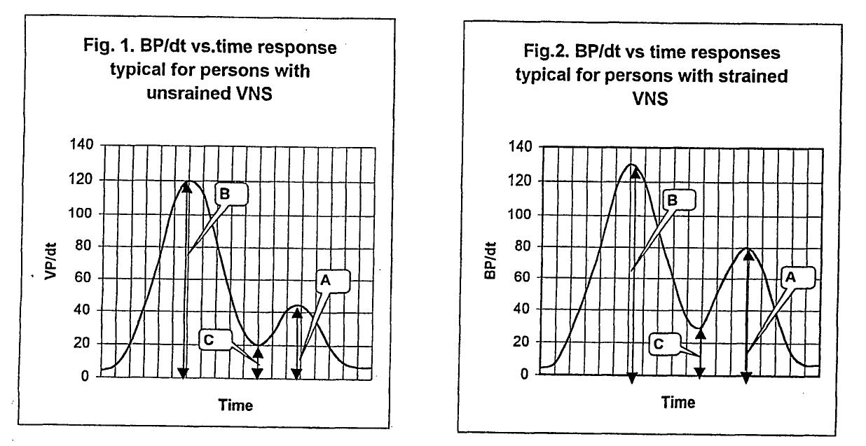

In FIGS. 1 and 2, typical BP/dt (with a constant component added) vs. time responses for a typical unstrained VNS and strained VNS are shown, respectively. BP/dt values were measured by a non-invasive means directly (not via differentiation of measured BP, for example). FIG. 2 corresponds to drugs abuse, in particular. Non-invasive BaTi differential pressure sensor was used. These Figs are that in the screen of a computer. This computer was used was used also as a source of dc voltage required for an amplifier built into the sensor. Depending upon power requirements, Com or USB port can be used. Analog-to-digit transformation of a signal from the sensor was produced in a computer with no separate or built into the sensor corresponding unit. No separate dc voltage source and analog-to-digit transforming unit reduces a cost of measurement device. Detailed description of a device used in present preferred embodiment is cited below.

The said in the above paragraph is true for all the measurements considered below.

One of the features of the present invention is VNSSF calculation and using in diagnosing. This parameter was found to be an easily detectable and a reliable parameter characterizing VNS state. In preferred embodiment of the present invention, calculations of VNSSF values and all the operations with them and/or other said parameters are made by a computer.

VNSSF according to the present invention is thus equal to:

I=(A−C)/(B−C),

where

I is VNSSF,

A is the BP/dt max. value corresponding to aorta push (contraction),

B is the BP/dt max. value corresponding to left heart ventricle push (contraction),

C is the BP/dt max. value corresponding to closing of aorta valve.

In FIGS. 1 and 2, A, B and C for the above expression are indicated giving I values 0.24 and 0.56, respectively.

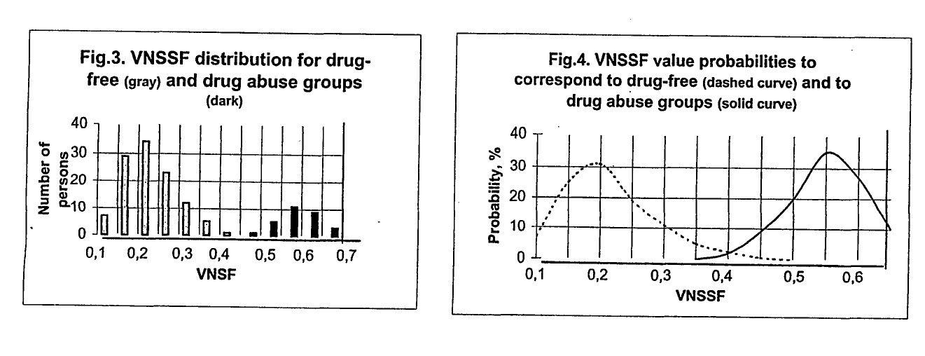

VNS state information according to the present invention was gathered from RG1 and drugs abuse (heroin) groups (RG2) in St.-Petersburg, Russia. VNSSF was determined from the gathered VNS state information. The data for VNSSF thus obtained are shown in FIG. 3. These data were treated to determine distribution law(s) of VNSSF for the said groups distribution. For those skilled in the art, it is evident, that with a particular distribution law (Gaussian random values distribution, for example) known, all probability estimates could be calculated or obtained directly from reference books. Thus, distribution law(s) determination gives a possibility to determine probability of a person's particular VNSSF value to fall within corresponding band of VNSSF, that is to determine quantitatively probability of a person's particular VNSSF value to correspond to strained or unstrained VNS state (drug abuse or drug-free groups, respectively, in the considered case). Determination of a distribution law and quantitative probability calculations were made by a computer.

In FIG. 4, illustrative responses for probabilities of a particular VNSSF value to correspond to that for drug-free and to that for drug abuse are shown. These responses are plotted using data of FIG. 3 for determination of VNSSF values distribution law and mathematical estimate of dissipation for the distribution law.

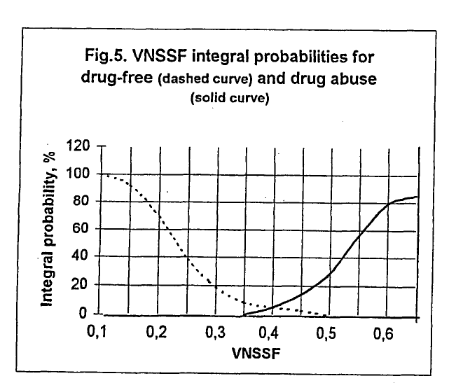

Data of FIG. 4 can be used for plotting the responses of FIG. 5, the latter show integrated probabilities values. For drug-free, integration is made in direction from infinite VNNSF value to 0. For drug abuse, integration is made in direction from 0 to Infinite VNSSF value.

What particular probability value is used further for determination of a cutoff VNSSF value, depends upon a method/device according to the present invention users' requirements. In case of practical separation of screened persons into two groups with drug-free taken out from further tests by other methods, the probability of false negative result (drug abuse is said to be drug-free) is of a primary importance. This probability value is usually to be as low as it is possible.

FIG. 5 gives a possibility to choose VNSSF cutoff level (separating VNSSFs to those for drug-free and for drugs abuse) to meet a method/device according to the present invention user's requirements expressed quantitatively.

Analysis of FIG. 5 shows that, for example,

with cutoff VNSSF value equal to 0.30, probability to false negative result is 0% with a reserve (0.35 value corresponds to 0%), and probability of false positive result (drug-free is said to be t drug abuse) equals 20%.

with cutoff VNSSF value equal to 0.35, a probability to false negative result is near 0%, and probability of false positive result equals 9%.

with cutoff VNSSF value equal to 0.40, a probability to false negative result equals 5%, and probability of false positive result equals 5% also.

Particular requirements of a user of a method/device according to the present invention can differ substantially depending upon specific screening (prescreening) requirements and a particular method/device to be further used. In case some other cheap method/device with very small probability of false positive result obtaining planned to be used after a method/device according to the present invention, choosing of VNSSF cutoff level equal 0.3 or even lower is substantiated. Indeed, it is not a problem to test further 20% again to confirm or to correct preceding test results and to obtain very near to zero false positive and false negative results, that is accuracy and specificity of test procedure (two said tests) at an acceptable level. In case some sophisticated expensive method/device is further used, each extra test can increase overall test procedure cost noticeably, and choosing of VNSSF cutoff level equal to 0.35 looks to be more reasonable (corresponding to 9% of persons to be further tested). In any way, false positive results probability seems to be predetermined with near zero value, thus maximal VNSSF cutoff level is 0.35.

As it can be seen from the above quantitative estimation, from 80% to 91% of tested persons could be taken off from further tests. Certainly, these figures can differ in practical cases for many reasons, including a share of drug abuses in screened volume, but substantial reducing of test procedure/device cost and improving of further used method/device characteristics if they are not satisfactory (false negative and/or false positive results probability) are evident.

It should be noted also that using in a method/device according to the present invention of other vascular (cardiovascular) parameters along with VNSSF, may improve quantitative estimates in a preferred embodiment. For example, heart rate measurements can take out of further tests those with high VNSSF but with bradycardia, because drug abuse is featured (at least heroin abuse) by expressed tachycardia.

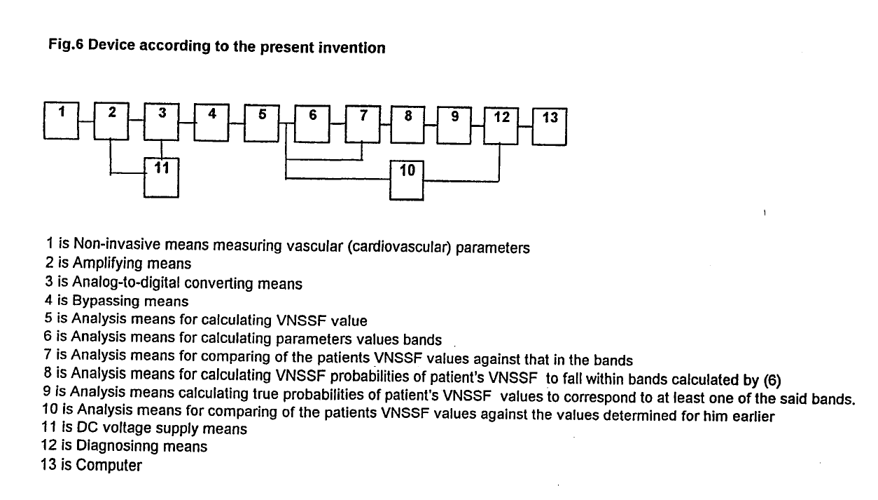

In FIG. 6, a flow chart of a device according to the present invention is shown. In FIG. 6 reference numeral 1 is a non-invasive means measuring vascular (cardiovascular) parameters, reference numeral 2 is an amplifying means, reference numeral 3 is an analog-to-digital converting means, reference numeral 4 is a bypassing means, reference numeral 5 is an analysis means for calculating VNSSF value, 6 is an analysis means for calculating parameters values bands, reference numeral 7 is an analysis means for comparing of the patients VNSSF values against that in the bands, reference numeral 8 is an analysis means for calculating VNSSF probabilities of patient's VNSSF to fall within bands calculated by (6), reference numeral 9 is an analysis means calculating true probabilities of patient's VNSSF values to correspond to at least one of the said bands, reference numeral 10 is an analysis means for comparing of the patients VNSSF values against the values determined for him earlier, reference numeral 11 is DC voltage supply means, reference numeral 12 is diagnosing means and reference numeral 13 is a computer.

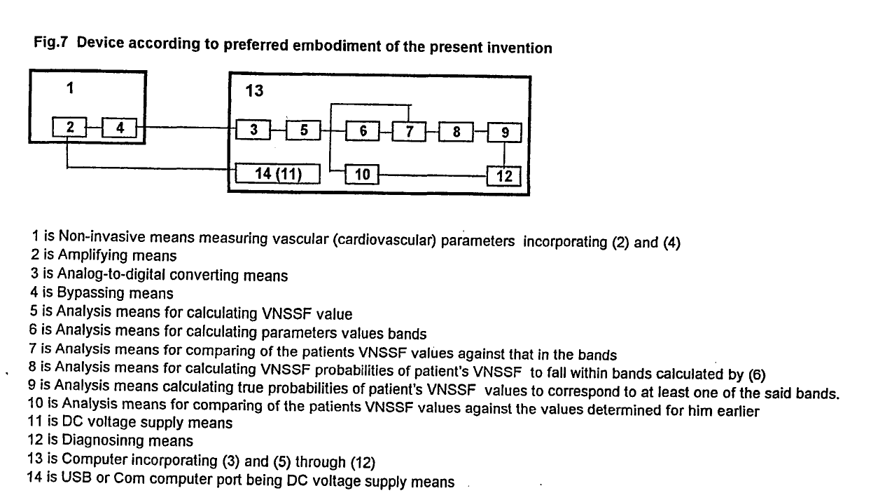

In FIG. 7, a flow chart of a device according to a preferred embodiment of the present invention is shown. In this device, a non-invasive means measuring vascular (cardiovascular) parameters incorporates amplifying and bypassing means 2 and 4; computer USB or Com port 14 serves as DC voltage supply means and this computer incorporates all the rest means in FIG. 6 using standard and special programs.

According to the present invention it is possible to connect a non-invasive means directly or via other means to an oscilloscope and/or to a computer.

The device of a preferred embodiment of the present invention comprises a converting means for analog-to-digital conversion of the signal from said non-invasive means. Said converting means may be incorporated with said non-invasive means. Preferably said converting means is a computer.

The device of a preferred embodiment of the present invention comprises also an amplifying means, said amplifying means being preferably incorporated with the said non-invasive means.

The device of a preferred embodiment of the present invention comprises also a bypassing means for blocking constant component of said non-invasive means signal from a computer.

The device of a preferred embodiment of the present invention comprises further a power supply means supplying dc voltage for said transforming means and/or for the said amplifying means, said power supply means being an oscilloscope or a computer.

Means for calculating VNSSF value is preferably incorporated with the said non-invasive means. Said calculating means is a computer and said analysis means for comparing of the patient's VNSSF and/or the said parameters values is also a computer.

Although the invention has been described in detail with reference to the illustrated preferred embodiments, variations and modifications exist within the scope and spirit of the invention as described and as defined in the following claims.

What is claimed is:

1. A method for diagnosing, monitoring and for using in treating of drugs and toxic substances abuse, alcoholism, drugs and other substances intoxication and hormone disorder due to a disease or natural processes in human organism comprising the steps of (1) gathering Vegetative Nervous System (VNS) state information through a non-invasive means from a reference group (RG1) consisting of persons with predetermined absence of drugs and toxic substances abuse, alcoholism, drugs and other substances intoxication and hormone disorder due to a disease or natural processes in human organism and reference groups (RG2) of patients with predetermined presence of drugs and toxic substances abuse, drugs and other substances intoxication and hormone disorder due to a disease or natural processes in human organism, (2) determining the VNS strain factors (VNSSF) from the gathered VNS state information and/or determining other vascular (cardiovascular) parameters, (3) determining the VNSSF and/or the said parameters values bands corresponding to the groups of step 1 above with inherent cutoff values limiting the bands, (4) comparing of the patient's VNSSF and/or the said parameters values against that within the bands of step (3) above, against limiting (cutoff) VNSSF and/or the said parameters values of these bands and against the patient's VNSSF and/or the said parameters values determined earlier, and (5) diagnosing the patient as belonging at least potentially to at least one of the groups (RG1 or RG2) of step (1) above or as having increasing or decreasing VNS strain in time.

2. The method of claim 1 being used for monitoring of patients VNS state in treating of diseases and in depression of human organism states, in case these diseases and states are featured by hormone disorder.

3. The method of claim 2 being used in treating of diseases and in depression of human organism states, in case these diseases and states are featured by hormone disorder, for evaluation of efficiency of treating methods and medicines used if this efficiency is indicated by increasing or decreasing of VNS strain, the latter characterized by VNSSF and/or other vascular (cardiovascular) parameters.

4. The method of claim 1 wherein the step of comparing patient's VNSSF and/or the said parameters values comprises the step of comparing against corresponding upper (cutoff) VNSSF and/or the said parameters value for RG1 band and against corresponding lower (cutoff) VNSSF and/or the said parameters value for RG2 band.

5. The method of claim 4 wherein the step of diagnosing the patient comprises the step of diagnosing the patient as having VNS state corresponding to no drug abuse, toxic substances abuse, drugs and toxic substances intoxication and hormone disorder due to a disease or natural processes in human organism in case patient's VNSSF and/or the said parameters values are below the said cutoff values.

6. The method of claim 4 wherein the step of diagnosing the patient comprises the steps of: (1) diagnosing the patient as having VNS state corresponding to drug abuse, toxic substances abuse, drugs intoxication, other substances intoxication and hormone disorder due to a disease or natural processes in human organism in case patient's VNSSF and/or the said parameters values exceed upper (cutoff) value in corresponding bands for RG1 and at least one lower (cutoff) value in corresponding bands for RG2 patients, and (2) specific diagnosing with a specific band (bands) with lower (cutoff) level being exceeded in view.

7. The method of claims 5 and 6 wherein probabilities of patient's VNSSF and/or the said parameters values to fall within the said bands is determined.

8. The method of claim 4 wherein the step of diagnosing the patient comprises the steps: (1) diagnosing the patient as having VNS state corresponding to undetermined situation requiring determination of probability values for patient's VNSSF and/or the said parameters values to correspond to at least one of said bands in case patient's VNSSF and/or the said parameters values exceed upper (cutoff) value in corresponding bands for RG1 and are below lower (cutoff) value in corresponding bands for RG2 patients, (2) using the probability values of step (1) for diagnosing the patient as having VNS state corresponding to that of claim 5 or claim 6 with a certain probability.

9. The method of claim 7 wherein the said probabilities are used for determination of patient's VNS state diagnosis true probability, thus to probability of correspondence of a patient's VNS to that for RG1 and for one or several groups for RG2

10. The method of claim 1 wherein the said vascular/cardiovascular parameter(s) is/are selected from a group comprising: total vascular conductance, vascular elasticity, large artery elasticity index, small artery elasticity index, vascular conductance and pulse rate or a combination thereof.

11. The method of claim 1 wherein the step of gathering VNS state information comprises the steps of: (1) affixing a non-invasive blood volume (BV) measuring probe and transducer means to the patient and (2) obtaining a data stream from the transducer means, the data stream including data for peaks and valleys of measured parameters within at least one full heart stroke period, at least peaks of the said parameters due to left heart ventricle and aorta and valley corresponding to aorta valve closing and/or including waveforms (measured BV vs time responses).

12. The method of claim 1 wherein the step of gathering VNS state information comprises the steps of: (1) affixing a non-invasive BV variation velocity (BV/dt) measuring probe and transducer means to the patient and (2) obtaining a data stream from the transducer means, the data stream including data for peaks and valleys of measured parameters within at least one full heart stroke period, at least peaks of the said parameters due to left heart ventricle and aorta and valley corresponding to aorta valve closing and/or including waveforms (BV/dt vs time responses).

13. The method of claim 1 wherein the step of gathering VNS state information comprises the steps of: (1) affixing a non-invasive blood pressure (BP) measuring probe and transducer means to the patient and (2) obtaining a data stream from the transducer means, the data stream including data for peaks and valleys of measured parameters within at least one full heart stroke period, at least peaks of the said parameters due to left heart ventricle and aorta and valley corresponding to aorta valve closing and/or including waveforms (BP vs time responses).

14. The method of claim 1 wherein the step of gathering VNS state information comprises the steps of: (1) affixing a non-invasive blood pressure variation velocity (BP/dt) measuring probe and transducer means to the patient and (2) obtaining a data stream from the transducer means, the data stream including data for peaks and valleys of measured parameters within at least one full heart stroke period, at least peaks of the said parameters due to left heart ventricle and aorta and valley corresponding to aorta valve closing and/or including waveforms (BP/dt vs time responses).

15. The method of claim 1 wherein the step of gathering VNS state Information comprises the steps of: (1) affixing a non-invasive BV variation acceleration (BV/dt2) measuring probe and transducer means to the patient and (2) obtaining a data stream from the transducer means, the data stream including data for peaks and valleys of measured parameters within at least one full heart stroke period, at least peaks of the said parameters due to left heart ventricle and aorta and valley corresponding to aorta valve closing and/or including waveforms (BV/dt2 vs time responses).

16. The method of claim 1 wherein the step of gathering VNS state information comprises the steps of: (1) affixing a non-invasive BP/dt2 measuring probe and transducer means to the patient and (2) obtaining a data stream from the transducer means, the data stream including data for peaks and valleys of measured parameters within at least one full heart stroke period, at least peaks of the said parameters due to left heart ventricle and aorta and valley corresponding to aorta valve closing and/or including waveforms (BP/dt2 vs time responses).

17. The method of claims 11 through 16 wherein the said measuring probe is selected from a group comprising: a photoplethysmograph probe, a physical pressure measuring probe, a rheograph probe; an ultrasonic probe and a X-ray probe or a combination thereof.

18. The method of claims 11 and 17 wherein blood volume BV vs time responses are transformed into BV/dt vs time responses by differentiation.

19. The method of claims 12 and 17 wherein BV/dt vs time responses are transformed into BV vs time responses by integration.

20. The method of claims 11 and 17 wherein BV vs time responses are transformed into BV/dt2 vs time responses by double differentiation.

21. The method of claims 12 and 17 wherein BV/dt vs time responses are transformed into BV/dt2 vs time responses by differentiation.

22. The method of claims 15 and 17 wherein BV/dt2 vs time responses are transformed into BV/dt vs time responses by integration.

23. The method of claims 15 and 17 wherein BV/dt2 vs time responses are transformed into BV vs time responses by double integration.

24. The method of claims 11 and 17 wherein BP vs time responses are transformed into BP/dt vs time responses by differentiation.

25. The method of claims 12 and 17 wherein BP/dt vs time responses are transformed into BP vs time responses by integration.

26. The method of claims 11 and 17 wherein BP vs time responses are transformed into BP/dt2 vs time responses by double differentiation.

27. The method of claims 12 and 17 wherein BP/dt vs time responses are transformed into BP/dt2 vs time responses by differentiation.

28. The method of claims 15 and 17 wherein BP/dt2 vs time responses are transformed into BP/dt vs time responses by integration

29. The method of claims 15 and 17 wherein BP/dt2 vs time responses are transformed into BP vs time responses by double integration.

30. The method of claims 11, 19 and 23 wherein VNSSF is determined using the following expression:
I=(A−C)/(B−C),
where
I is VNSSF,
A is the BV corresponding to aorta push (contraction),
B is the BV corresponding to left heart ventricle push (contraction),
C is the BV corresponding to closing of aorta valve.

31. The method of claim 30 wherein A and B are the maximal values and C is the minimal value of corresponding BV.

32. The method of claims 13, 25 and 29 wherein VNSSF is determined using the following expression:
I=(A−C)/(B−C),
where
I is VNSSF,
A is the BP corresponding to aorta push (contraction),
B is the BP corresponding to left heart ventricle push (contraction),
C is the BP corresponding to closing of aorta valve.

33. The method of claim 32 wherein A and B are the maximal values and C is the minimal value of corresponding BP.

34. The method of claims 12, 18 and 22 wherein VNSSF is determined using the following expression:
I=(A−C)/(B−C),
where
I is VNSSF,
A is the BV/dt corresponding to aorta push (contraction),
B is the BV/dt corresponding to left heart ventricle push (contraction),
C is the BV/dt corresponding to closing of aorta valve.

35. The method of claim 34 wherein A and B are the maximal values and C is the minimal value of corresponding BV/dt.

36. The method of claim 14, 24 and 28 wherein VNSSF is determined using the following expression:
I=(A−C)/(B−C),
where
I is VNSSF,
A is the BP/dt corresponding to aorta push (contraction),
B is the BP/dt corresponding to left heart ventricle push (contraction),
C is the BP/dt corresponding to closing of aorta valve.

37. The method of claim 36 wherein A and B are the maximal values and C is the minimal value of corresponding BP/dt.

38. The method of claims 15, 20 and 21 wherein VNSSF is determined using the following expression:
I=(A−C)/(B−C),
where
I is VNSSF,
A is the BV/dt2 corresponding to aorta push (contraction),
B is the BV/dt2 corresponding to left heart ventricle push (contraction),
C is the BV/dt2 corresponding to closing of aorta valve.

39. The method of claim 38 wherein A and B are the maximal values and C is the minimal value of corresponding BV/dt2 velocities.

40. The method of claims 16, 26 and 27 wherein VNSSF is determined using the following expression:
I=(A−C)/(B−C),
where
I is VNSSF,
A is the BP/dt2 corresponding to aorta push (contraction),
B is the BP/dt2 corresponding to left heart ventricle push (contraction),
C is the BP/dt2 corresponding to closing of aorta valve.

41. The method of claim 40 wherein A and B are the maximal values and C is the minimal value of corresponding BP/dt2.

42. The method of Determination of VNS State including measuring of Pulse wave, its mathematical treatment and using the following expression
I=(A−C)/(B−C)
where
A is the velocity of aorta push
B is the velocity of left ventricle contraction and
C is the minimal velocity of blood pressure variation corresponding to
closing of aorta valve,
for determination of I, that is VNSSF, whereas the said factor value exceeding 0.3, being measured at maximal A and B values, corresponds to strained VNS state due to hormone disorder caused by alcohol and/or drugs and/or toxic substances and/or diseases of vegetative nervous (vascular) system.

43. The method of claim 17 wherein the said data stream is fed from the said transducer means to an oscilloscope and/or to a computer.

44. The method of claim 17 wherein analog-to-digital conversion of the said data stream is produced.

45. The method of claim 44 wherein the said analog-to-digital conversion is produced by one of the following means: a separate unit, a functional unit built into the said probe and a computer.

46. The method of claim 17 wherein the said data stream signal is amplified.

47. The method of claim 46 wherein the said data stream signal is amplified by one of the following means: a separate unit or a functional unit built into the said probe.

48. The method of 43 wherein constant component of a signal from the said probe is bypassed from said oscilloscope and/or computer.

49. The method of claims 43 through 48 wherein dc voltage required for the said analog-to-digital conversion and/or for the said data stream signal to be amplified is supplied from one of the following means: a separate unit, oscilloscope and a computer.

50. The method of claims 18, 21, 24 and 27 wherein the said differentiation is produced by one of the following means: by a separate differentiating unit following the said non-invasive probe, by a functional differentiating unit built into the said non-invasive probe, or by a computer by program means.

51. The method of claims 19, 22, 25 and 28 wherein the said integration is produced by one of the following means: by a separate integrating unit following the said non-invasive probe, by a functional integrating unit built into the said non-invasive probe or by a computer by program means.

52. The method of claims 20 and 26 wherein the said double differentiation is produced by one of the following means: by a separate double differentiating unit or by two separate differentiating units following the said non-invasive probe, by a functional double differentiating unit or by two functional differentiating units built into the said non-invasive probe, or by a computer by program means.

53. The method of claims 23 and 29 wherein the said double integration is produced by one of the following means: by a separate double integrating unit or by two separate integrating units following the said non-invasive probe, by a functional double integrating unit or by two functional integrating units built into the said non-invasive probe, or by a computer by program means.

54. A non-invasive device for diagnosing, monitoring and for using in treating of diseases and states, in particular of drugs and toxic substances abuse, alcoholism, drugs and other substances intoxication and hormone disorder due to a disease or natural processes in human organism, said device comprising (1) a non-invasive means measuring vascular (cardiovascular) parameters characterizing VNS state, (2) analysis means for calculating of VNSSF values from the measured vascular (cardiovascular) parameters values, (3) analysis means for calculating (determination) of VNSSF and/or the said parameters values bands corresponding to different measurements series with inherent cutoff values limiting the bands, (4) analysis means for comparing of the patient's VNSSF and/or the said parameters values against that within the bands in (3) above, against limiting (cutoff) VNSSF and/or the said parameters values of these bands and against the patient's VNSSF and/or the said parameters values determined earlier, and (5) analysis means for diagnosing the patient as corresponding at least potentially to at least one of the said measurements series or as having increasing or decreasing VNS strain in time.

55. The device of claim 54 further including analysis means calculating probabilities of patient's VNSSF and/or the said parameters values to fall within the said bands.

56. The device of claim 54 further including analysis means calculating true probabilities of patient's VNSSF and/or the said parameters values to correspond to at least one of the said bands.

57. The device of claim 54 wherein the said non-invasive means is non-invasive means measuring at least one of the following group of vascular cardiovascular parameters: total vascular conductance, vascular elasticity, large artery elasticity index, small artery elasticity index systemic vascular conductance pulse rate, or a combination thereof.

58. The device of claim 54 wherein the said non-invasive means is a non-invasive means measuring BV peaks and valleys within at least one full heart stroke period, at least peaks of BV due to left heart ventricle and aorta and valley corresponding to aorta valve closing and/or BV waveforms (BV vs time responses).

59. The device of claim 54 wherein the said non-invasive means is a non-invasive means measuring BV/dt peaks and valleys within at least one full heart stroke period, at least peaks of BV/dt due to left heart ventricle and aorta and valley corresponding to aorta valve closing and/or BV/dt waveforms (BV/dt vs time responses).

60. The device of claim 54 wherein the said non-invasive means is a non-invasive means measuring BP. peaks and valleys within at least one full heart stroke period, at least peaks of BP due to left heart ventricle and aorta and valley corresponding to aorta valve closing and/or BP waveforms (BP vs time responses).

61. The device of claim 54 wherein the said non-invasive means is a non-invasive means measuring BP/dt peaks and valleys within at least one full heart stroke period, at least peaks of BP/dt due to left heart ventricle and aorta and valley corresponding to aorta valve closing and/or BP/dt waveforms (BP/dt vs time responses).

62. The device of claim 54 wherein the said non-invasive means is a non-invasive means measuring BV/dt2 peaks and valleys within at least one full heart stroke period, at least peaks of BV/dt2 due to left heart ventricle and aorta and valley corresponding to aorta valve closing and/or BV/dt2 waveforms (BV/dt2 vs time responses).

63. The device of claim 54 wherein the said non-invasive means is a non-invasive means measuring BP/dt2 peaks and valleys within at least one full heart stroke period, at least peaks of BP/dt2 due to left heart ventricle and aorta and valley corresponding to aorta valve closing and/or BP/dt2 waveforms (BP/dt2 vs time responses).

64. The device of claims 58 through 63 wherein the said non-invasive means is selected from the following group: a photoplethysmograph probe, a physical pressure measuring, a rheograph probe, an ultrasonic probe, and an X-ray probe.

65. The device of claims 58 and 64 further including calculating means transforming BV vs time responses into BV/dt vs time responses by differentiation.

66. The device of claims 59 and 64 further including calculating means transforming BV/dt vs time responses into BV vs time responses by integration.

67. The device of claims 58 and 64 further including calculating means transforming BV vs time responses into BV/dt2 vs time responses by double differentiation.

68. The device of claims 59 and 64 further Including calculating means transforming BV/dt vs time responses into BV/dt2 vs time responses by differentiation.

69. The device of claims 62 and 64 further including calculating means transforming BV/d2 vs time responses into BV/dt vs time responses by integration.

70. The device of claims 62 and 64 further including calculating means transforming BV/d2 vs time responses into BV vs time responses by double integration.

71. The device of claims 60 and 64 further including calculating means transforming BP vs time responses into BP/dt vs time responses by differentiation.

72. The device of claims 61 and 64 further including calculating means transforming BP/dt vs time responses into BP vs time responses by integration.

73. The device of claims 60 and 64 further including calculating means transforming BP vs time responses into BP/dt2 vs time responses by double differentiation.

74. The device of claims 61 and 64 further including calculating means transforming BP/dt vs time responses into BP/dt2 vs time responses by differentiation.

75. The device of claims 63 and 64 further including calculating means transforming BP/dt2 vs time responses into BP/dt vs time responses by integration.

76. The device of claims 63 and 64 further including calculating means transforming BP/dt2 vs time responses into BP vs time responses by double integration.

77. The device of claims 58, 66 and 70 wherein the said analysis means for calculating of the VNSSF value from the measured vascular (cardiovascular) parameters values calculates this value according to the following expression:
I=(A−C)/(B−C),
where
I is VNSSF,
A is the BV corresponding to aorta push (contraction),
B is the BV corresponding to left heart ventricle push (contraction),
C is the BV corresponding to closing of aorta valve.

78. The device of claim 77 wherein A and B are the maximal values and C is the minimal value of corresponding BV.

79. The device of claims 60, 72 and 76 wherein the said analysis means for calculating of the VNSSF value from the measured vascular (cardiovascular) parameters values calculates this value according to the following expression:
I=(A−C)/(B−C),
where
I is VNSSF,
A is the BP corresponding to aorta push (contraction),
B is the BP corresponding to left heart ventricle push (contraction),
C is the BP corresponding to closing of aorta valve.

80. The device of claim 79 wherein A and B are the maximal values and C is the minimal value of corresponding BP.

81. The device of claims 59, 65 and 69 wherein the said analysis means for calculating of the VNSSF value from the measured vascular (cardiovascular) parameters values calculates this value according to the following expression:
I=(A−C)/(B−C),
where
I is VNSSF,
A is the BV/dt corresponding to aorta push (contraction),
B is the BV/dt corresponding to left heart ventricle push (contraction),
C is the BV/dt corresponding to closing of aorta valve.

82. The device of claim 81 wherein A and B are the maximal values and C is the minimal value of corresponding BV/dt.

83. The device of claims 61, 71 and 75 wherein the said analysis means for calculating of the VNSSF value from the measured vascular (cardiovascular) parameters values calculates this value according to the following expression:
I=(A−C)/(B−C),
where
I is VNSSF,
A is the BP/dt corresponding to aorta push (contraction),
B is the BP/dt corresponding to left heart ventricle push (contraction),
C is the BP/dt corresponding to closing of aorta valve.

84. The device of claim 83 wherein A and B are the maximal values and C is the minimal value of corresponding BP/dt.

85. The device of claims 62, 67 and 68 wherein the said analysis means for calculating of the VNSSF value from the measured vascular (cardiovascular) parameters values calculates this value according to the following expression:
I=(A−C)/(B−C),
where
I is VNSSF,
A is the BV/dt2 corresponding to aorta push (contraction),
B is the BV/dt2 corresponding to left heart ventricle push (contraction),
C is the BV/dt2 corresponding to closing of aorta valve.

86. The device of claim 85 wherein A and B are the maximal values and C is the minimal value of corresponding BV/dt2.

87. The device of claims 63, 73 and 74 wherein the said analysis means for calculating of the VNSSF value from the measured vascular (cardiovascular) parameters values calculates this value according to the following expression:
I=(A−C)/(B−C),
where
I is VNSSF,
A is the BP/dt2 corresponding to aorta push (contraction),
B is the BP/dt2 corresponding to left heart ventricle push (contraction),
C is the BP/dt2 corresponding to closing of aorta valve.

88. The device of claim 87 wherein A and B are the maximal values and C is the minimal value of corresponding BP/dt2.

89. The device of claim 54 wherein the said analysis means for diagnosing the patient, diagnosis VNNSF exceeding 0.3, being measured at maximal A and B values, as corresponding to strained VNS state due to drugs and toxic substances abuse, alcoholism, drugs and other substances intoxication and hormone disorder due to a disease or natural processes in human organism.

90. The device of claim 64 wherein said non-invasive means is connected directly or via other means to an oscilloscope and/or directly or via other means to a computer.

91. The device of claim 64 further comprising a converting means for analog-to-digital conversion of the signal from the said non-invasive means.

92. The device of claim 91 wherein the said converting means is incorporated with the said non-invasive means.

93. The device of claim 91 wherein the said converting means is a computer.

94. The device of claim 64 further comprising an amplifying means.

95. The device of claim 94 wherein the said amplifying means is incorporated with the said non-invasive means.

96. The device of claim 64 and 90 further comprising a bypassing means for blocking constant component of the said non-invasive means signal from the said oscilloscope and/or computer.

97. The device of claims 91 through 95 further comprising a power supply means supplying dc voltage for the said converting means and/or for the said amplifying means, said dc voltage supplying means being selected from the following group: an oscilloscope or a computer.

98. The device of claims 65 through 76 wherein the said calculating means is incorporated with the said non-invasive means.

99. The device of claims 65 through 76 wherein the said calculating means is a computer.

100. The device of claim 54 wherein a computer is used as said analysis means for calculating (determination) of VNSSF and/or the said parameters values bands, and/or for comparing of the patient's VNSSF and/or the said parameters values and/or for diagnosing the patient.
